(19) United States
(12) Patent Application Publication
(10) Pub. No.: US 2024/0180481 A1
(43) Pub. Date: Jun. 6, 2024

(54) DEVICE TO SUPPORT DERMATOLOGICAL DIAGNOSIS

(71) Applicant: Inpeco SA, Novazzano (CH)

(72) Inventors: Gianandrea PEDRAZZINI, Novazzano (CH); Gastone CIUTI, Pisa (IT); Tommaso MELILLO, Pisa (IT); Tommaso BANFI, Pisa (IT); Arianna CIGNONI, Pisa (IT); Marco DAL CANTO, Pisa (IT); Alessia BLUNDO, Pisa (IT)

(21) Appl. No.: 18/285,761

(22) PCT Filed: Apr. 6, 2022
(86) PCT No.: PCT/IB2022/053197

§ 371 Date: Oct. 5, 2023

(30) Foreign Application Priority Data
Apr. 7, 2021 (IT) 102021000008639

(51) Int. Cl. A61B 5/00; G16H 30/40

(52) U.S. Cl. CPC A61B 5/444; A61B 5/0075; A61B 5/0077; A61B 5/0082; A61B 5/7267; A61B 5/7425; G16H 30/40

(57) ABSTRACT

A device to support dermatological diagnosis for recognition and evaluation of a skin lesion, includes a vision module including a sensor for acquiring an image of the skin lesion; a spectroscopy module including a spectroscope for acquiring a spectral response of the skin lesion in a visible and infrared band; an electronic control unit configured for storing data acquired by said modules, including images acquired by said vision module and spectral responses acquired by said spectroscopy module, for sending said acquired data to an external processing system for processing by an internal processing system (SI), for receiving an objective evaluation result of the skin lesion processed by said processing system, as a result of integration of the acquired data performed by said processing system and for showing said objective evaluation result on a display screen.

### FIELD OF THE INVENTION

The present invention relates to a device to support dermatological diagnosis for the recognition of skin lesions, in particular to evaluate the nature of skin lesions.

### PRIOR ART

Nowadays, dermatoscopy is often used to carry out diagnostic investigations of tumor-like skin lesions, which is a non-invasive technique aimed at the early diagnosis of skin tumors or other skin lesions, based on an optical instrument called dermatoscope. Typically, the dermatoscope is a manual instrument including a lens, able to provide enlargements of the skin area to be analyzed, specifically illuminated with an incident light. This approach based on visual inspections is highly dependent on the experience of the clinical operator, as it is exclusively based on the recognition of some specific characteristics of the lesion.

There are also multiple techniques for the classification of skin lesions based on different physical principles, relating for example to reflectance spectroscopy in the visible and near infrared bands.

The present invention starts from the desire to develop a device of the type indicated at the beginning of the present description, arranged to provide objective information on the inspected lesion, in order to obtain a particularly precise characterization thereof, optimizing the dermatological diagnosis operations, so as to decrease the variability linked to the experience of the medical operator.

An example of a system arranged to provide diagnostic information of a tumor skin lesion is described in the document RU 2506049 C1. However, the solutions known so far have various drawbacks, including a high operating complexity and/or poor precision to characterize the inspected lesion, which is why it is intended to propose an alternative solution that meets the above requirements.

### OBJECT OF THE INVENTION

It is therefore an object of the invention is to provide a device to support dermatological diagnosis for the recognition of skin lesions, in particular to evaluate the possibly malignant nature of said skin lesion, which overcomes the aforementioned drawbacks and is particularly efficient in guaranteeing objective and quantitative information on the analyzed skin lesion, in order to obtain an extremely precise and reliable characterization.

A further object of the invention is to provide a device arranged to provide a single particularly significant output of the nature of the analyzed lesion.

A further object of the invention is to achieve the aforementioned objectives with a device which is simple and intuitive to use.

### SUMMARY OF THE INVENTION

In view of the achievement of the aforementioned objects, the present invention relates to a device to support dermatological diagnosis for the recognition of a skin lesion, in particular to evaluate the nature of the lesion, said device comprising:
a vision module including at least one sensor able to acquire an image of a skin lesion of a patient,
a spectroscopy module including at least one spectroscope able to acquire a spectral response of the skin lesion in a determined visible and infrared band,
at least one housing including said vision module and said spectroscopy module,
at least one electronic control unit configured for:
controlling said vision module and said spectroscopy module,
storing a plurality of data acquired by means of said modules, in particular one or more images acquired by means of said vision module and one or more spectral responses acquired by means of said spectroscopy module,
sending said acquired data to a processing system,
receiving an objective evaluation result of the skin lesion processed by said processing system, as a result of the integration of the acquired data performed by said processing system, and
showing said objective evaluation result on a display screen,
in such a way that said device is configured to acquire a plurality of input data on the skin lesion to be analyzed, based on different physical principles, and to allow the visualization of a single output result representative of objective information about the characteristics of the skin lesion of interest.

Preferably, at least one sensor is an RGB camera sensitive in the visible frequencies (430-770 THz), able to acquire an image of the lesion, and said vision module further comprises:
a lighting system consisting of a plurality of LED devices configured to illuminate an area of a patient's skin including the lesion to be analyzed, and
at least one polarization filter able to allow a visualization of the deepest layers of the skin, eliminating surface reflections due to the reflection of light on the corneal layer of the patient's skin.

Still according to a preferred feature of the invention, a spacer element is associated with said vision module, said spacer element being able to define, during the use of the device, a known distance from an area of the skin including the lesion to be analyzed, in order to ensure a focusing of the area framed by said sensor. Such spacer element can also be moved axially by means of an adjustment device comprising a threaded coupling and an elastic backlash take up system.

Still according to a preferred feature of the invention, said spectroscopy module further comprises:
a light source able to generate a light signal with a given intensity of reflection measurable by said spectroscope after the interaction with the skin area including the lesion to be analyzed,
a connection system configured to bring the light signal generated by the light source to the patient's skin, and to intercept the reflected signal after the interaction with the lesion of interest, in order to transmit it towards said spectroscope, and
a system comprising one or more specific optical elements for the focusing and dispersion of the light beam to be analyzed.

The lighting sources of said vision and spectroscopy modules can be separate or integrated and thus constitute a single lighting source adequate for multiple modules, in order to facilitate the optimization of space, consumption and performance.

The device according to the invention can comprise further sensors based on physical principles different from those of the vision and spectroscopy modules, so as to further strengthen the precision of the final output classification of the analyzed lesion, said further sensors comprising one or more of the following modules: a thermography module including one or more thermal cameras to obtain one or more thermographic images of the lesion, an impedance analyzer, a vision module operating in the infrared SWIR (“Short Wave Infrared”, 1000-2500 nm) region to obtain images that emphasize the vascularization of the area including the lesion.

The present invention is also directed to a system to support dermatological diagnosis for the recognition of skin lesions, comprising a device having the aforementioned characteristics and a data processing system able to receive data acquired by said device and to process a final informative result of the analyzed lesion.

Such processing system can be internal, external or a combination of the two depending on the availability or not of an internet connection in the areas affected by the information flow of the system itself (for example, the different rooms of a laboratory).

Further features of the invention are indicated in the attached claims and in the following description.

### DETAILED DESCRIPTION OF PREFERRED EMBODIMENTS

Further characteristics and advantages of the present invention will become clearer from the following description, with reference to the attached drawings provided purely by way of non-limiting example, in which:

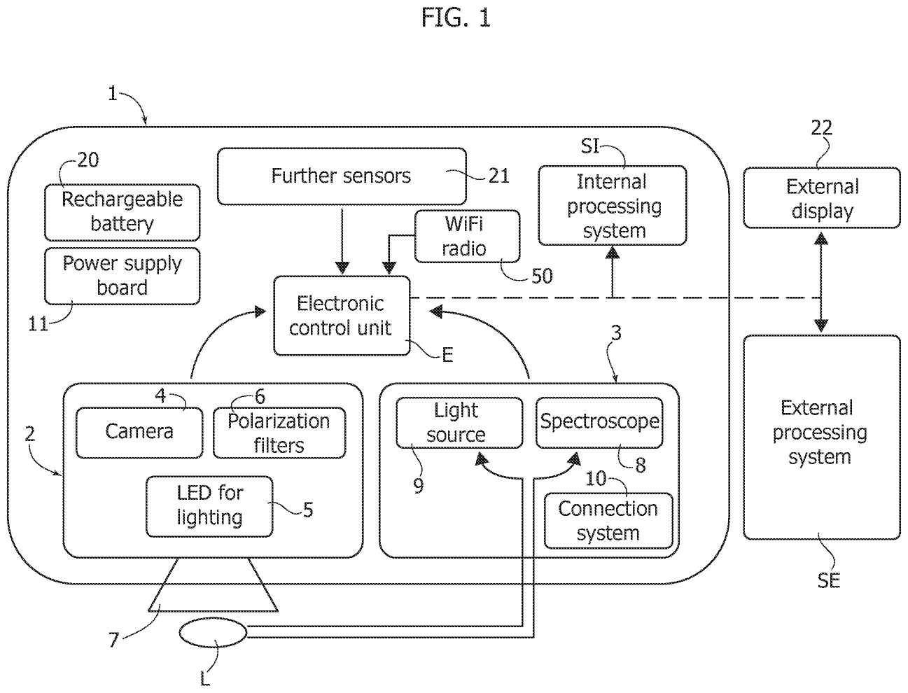

FIG. 1 is a schematic representation of the device according to the present invention;

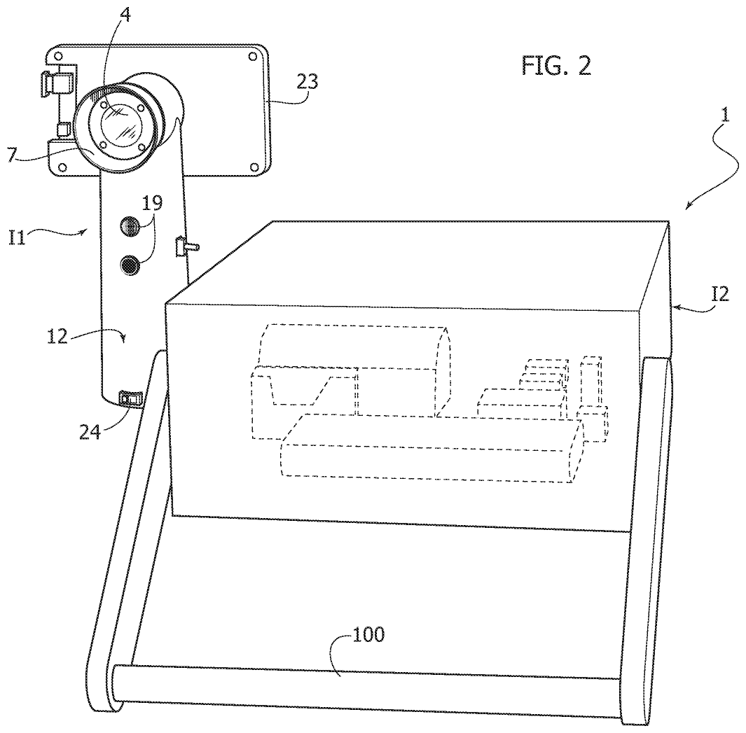

FIG. 2 is a perspective view of a first embodiment of the device schematically illustrated in the previous figure;

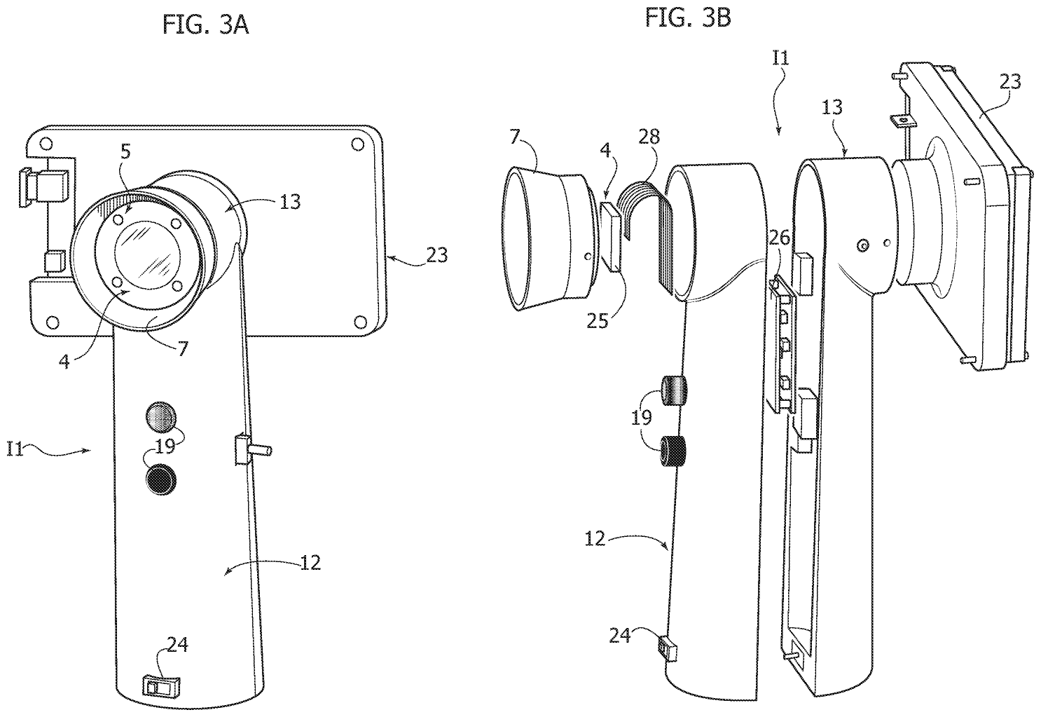

FIGS. 3A, 3B are a perspective view and an exploded perspective view of a vision module belonging to the device of the invention, according to the first embodiment illustrated in FIG. 2,

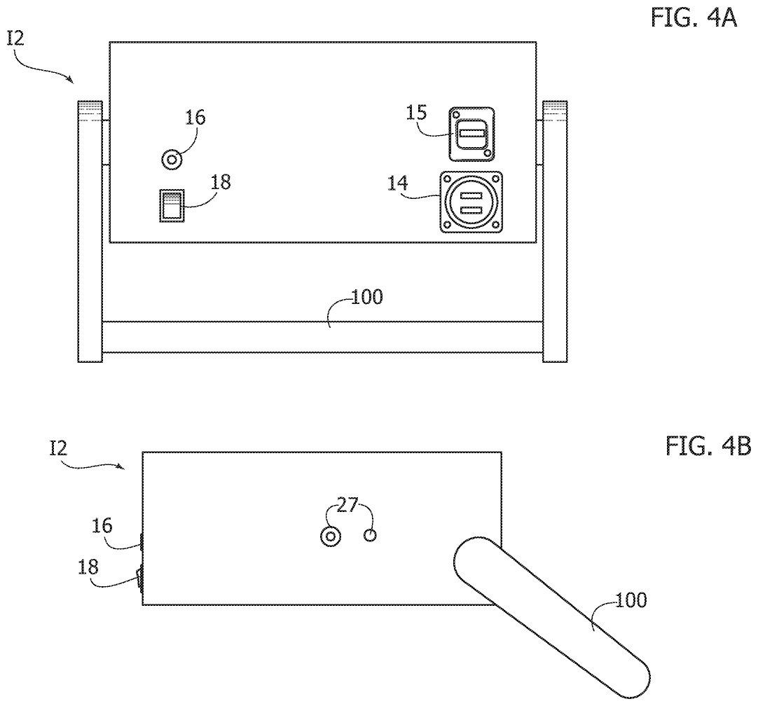

FIGS. 4A, 4B illustrate two side views of a spectroscopy module belonging to the device of the invention, in the first embodiment illustrated in FIG. 2,

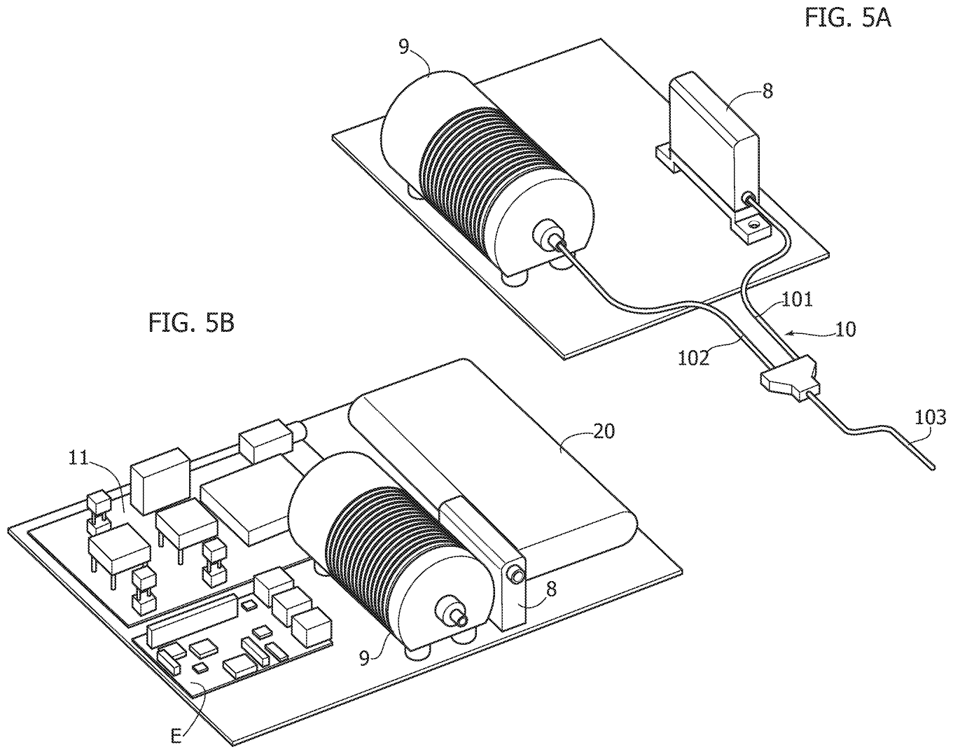

FIGS. 5A, 5B are perspective views illustrating the internal components of the housing of the spectroscopy module of the device exemplified in the previous figures,

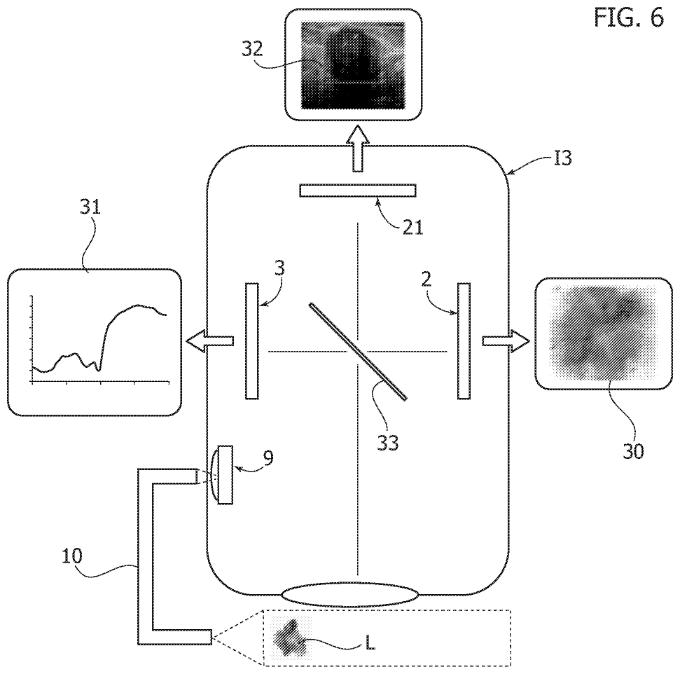

FIG. 6 schematically illustrates a second embodiment of a device according to the present invention,

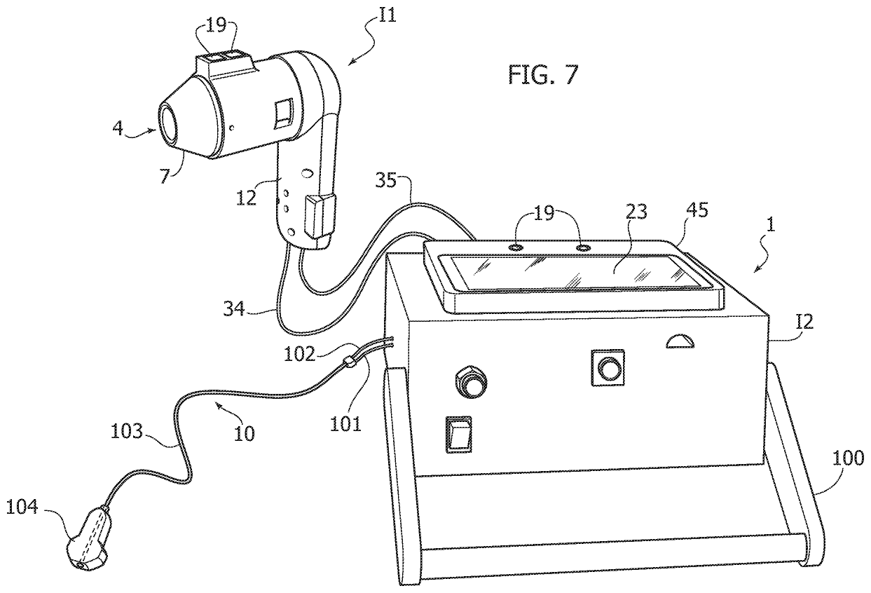

FIG. 7 is a variation of FIG. 2, which illustrates a third embodiment of the device of the invention,

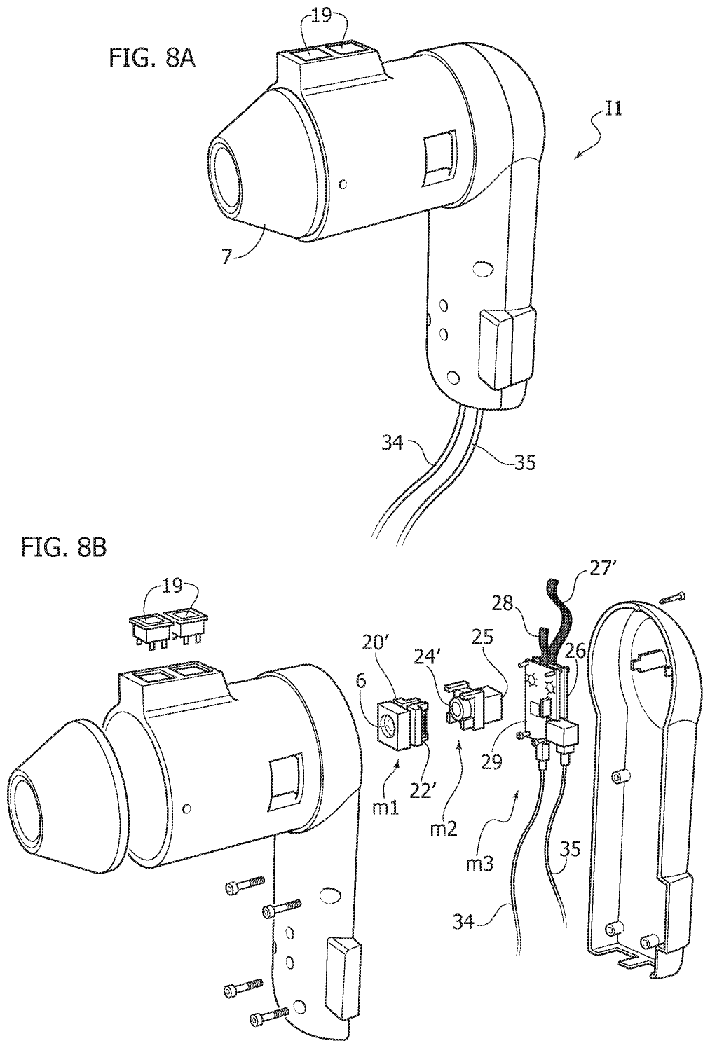

FIGS. 8A, 8B are variations of FIGS. 3A, 3B, which illustrate a perspective view and an exploded perspective view of a vision module belonging to the device of the invention, according to the third embodiment illustrated in FIG. 7,

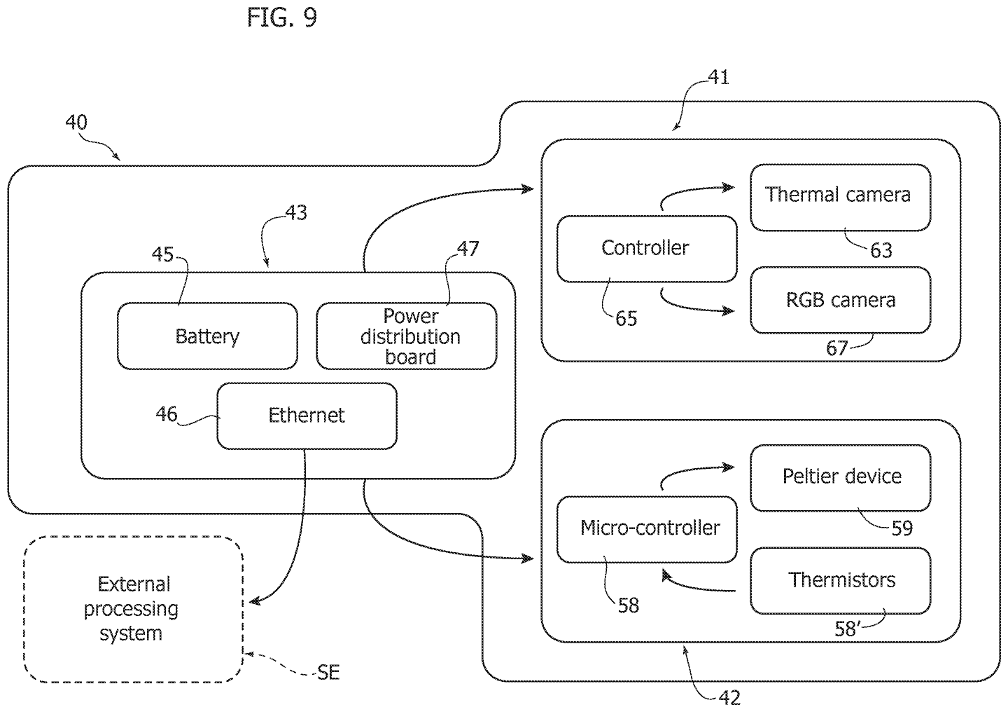

FIG. 9 is a schematic representation of the device according to a further embodiment comprising a thermography module;

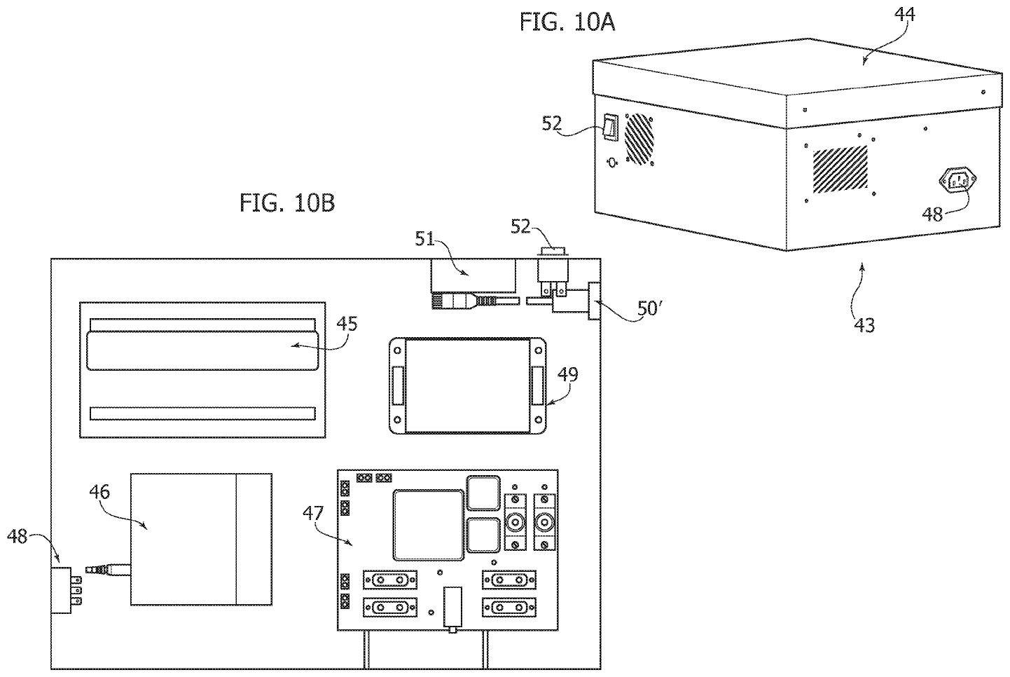

FIGS. 10A, 10B are a perspective view and a section view of a power supply sub-module belonging to the device of the invention, according to the embodiment illustrated in FIG. 9,

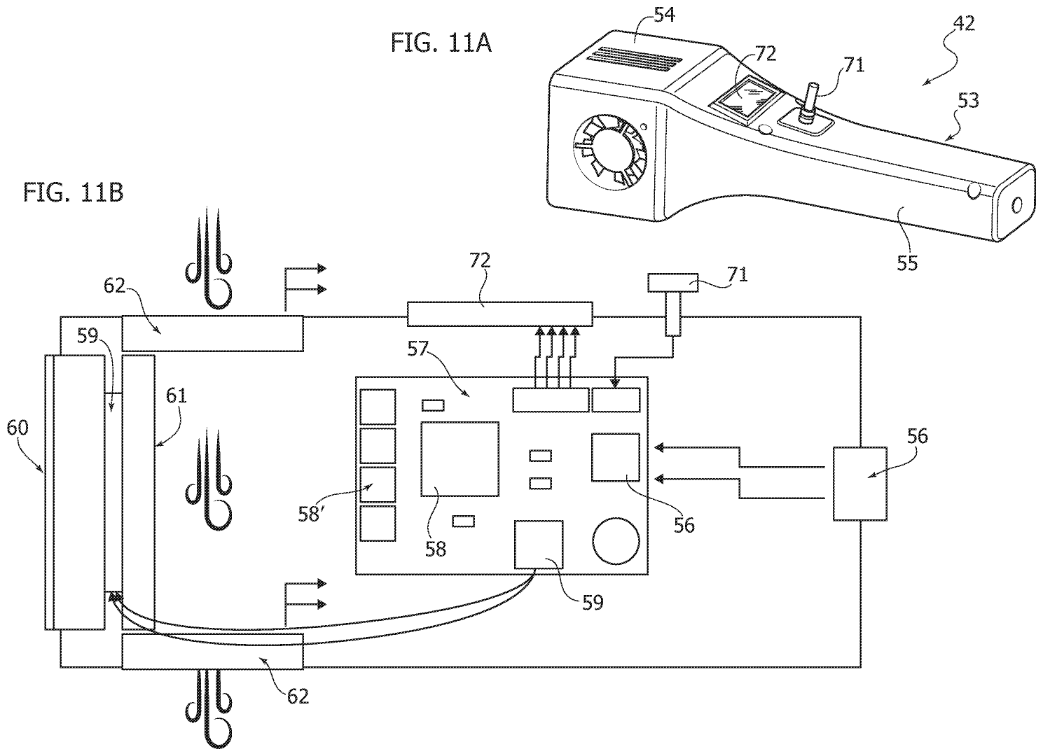

FIGS. 11A, 11B are a perspective view and a schematic view of an interaction thermal instrument belonging to the device of the invention, according to the embodiment illustrated in FIG. 9,

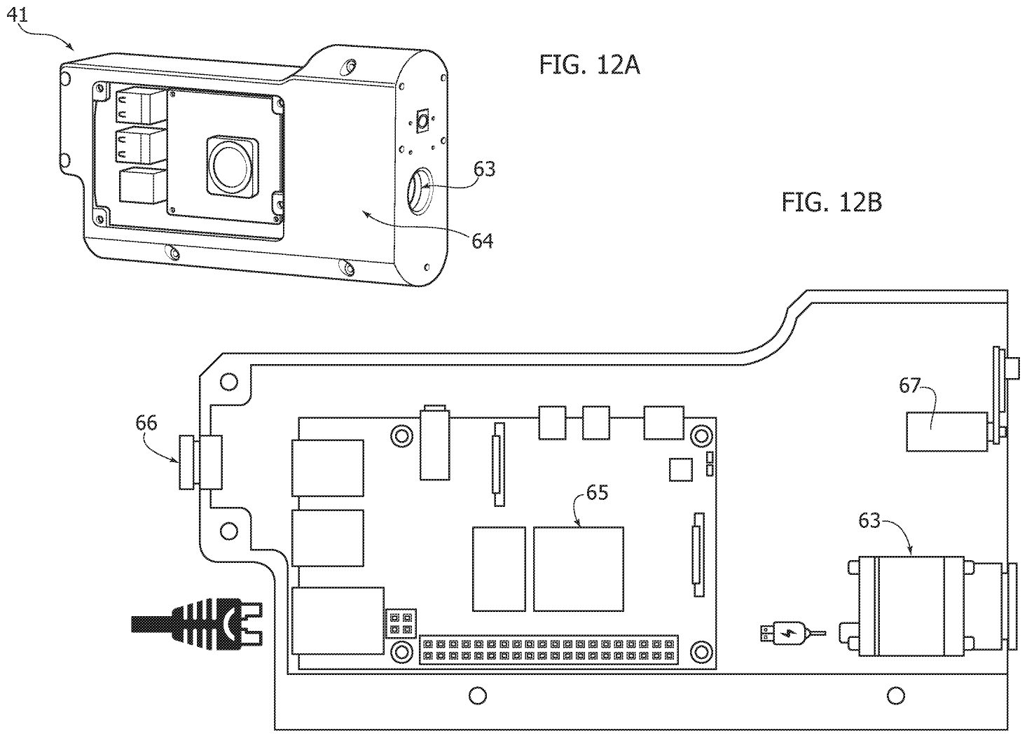

FIGS. 12A, 12B are a perspective view and a schematic view of a thermal sub-module belonging to the device of the invention, according to the embodiment illustrated in FIGS. 9, and

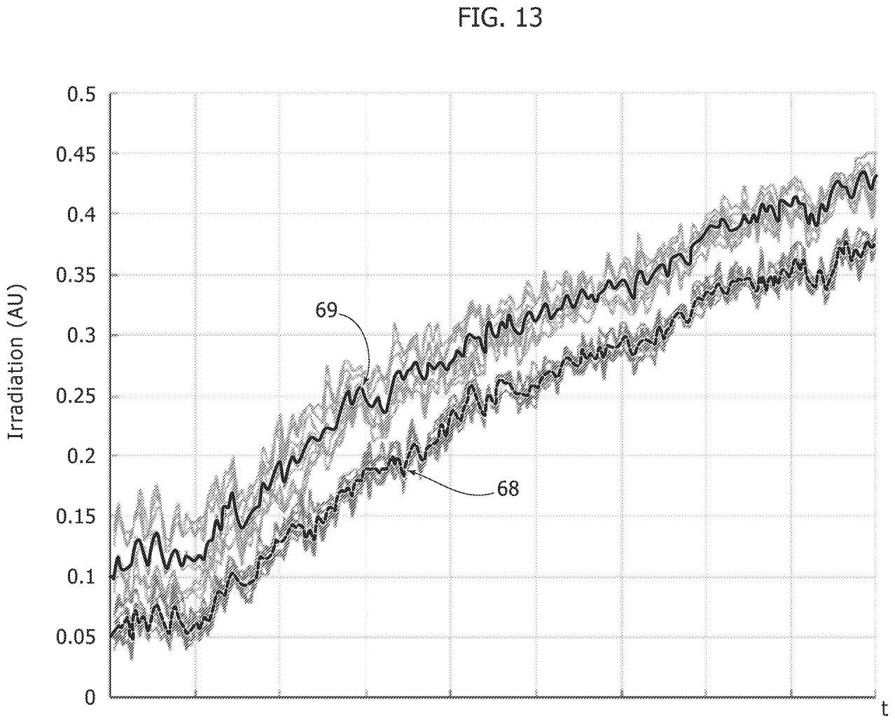

FIG. 13 is a graph showing the thermal recovery of a healthy tissue region and of a tumor tissue region after a local stimulation carried out with the device of the invention, according to the embodiment illustrated in FIG. 9.

The following description illustrates various specific details aimed at an in-depth understanding of examples of one or more embodiments. The embodiments can be made without one or more of the specific details, or with other methods, components, materials, etc. In other cases, known structures, materials or operations are not shown or described in detail to avoid obscuring the various aspects of the embodiments. Reference to “an embodiment” within this description means that a particular configuration, structure or feature described in connection with the embodiment is included in at least one embodiment. Hence, phrases such as “in an embodiment”, possibly present in different places in this description, are not necessarily referred to the same embodiment. Furthermore, particular shapes, structures or features can be combined in an adequate way in one or more embodiments and/or associated with the embodiments in a different way from as illustrated here, whereby for example a feature here exemplified according to a figure can be applied to one or more embodiments exemplified in a different figure.

The references illustrated herein are for convenience only and therefore do not limit the scope of protection or the extent of the embodiments.

It should be noted that the shapes of the device exemplified in the figures are not to be considered in any way limiting, but merely representative of some of the many embodiments of a device to support dermatological diagnosis incorporating the peculiar characteristics of the invention.

In the annexed drawings, reference 1 indicates as a whole a dermatological diagnosis device for the recognition of tumor skin lesions, in particular for performing an evaluation on the possibly malignant nature of the analyzed skin lesion. The device 1 is therefore inserted in the context of the screening dynamics of skin lesions (both melanoma and non-melanoma) in the clinical field.

The device 1 according to the invention is configured to provide objective and quantitative information on the analyzed tumor skin lesion, in order to have a precise and reliable support to diagnose the lesion and decrease the variability of a correct diagnosis, often linked to the experience of the medical staff. Of course, the device 1 is able to give a diagnostic support to the dermatologist and does not in any way aim to replace qualified experts to ensure the health of patients.

As will be evident from the following description, the device 1 according to the invention integrates a plurality of different technologies, designed to provide different information on the inspected lesion, in order to obtain an extremely precise characterization thereof.

The following description will now describe the different components of the device 1.

As shown in FIG. 1, representative of a schematic view of the device of the invention, the device 1 comprises a vision module 2, a spectroscopy module 3, possibly further sensors 21 and an electronic control unit E able to control the vision module 2, the spectroscopy module 3 and the possible further sensors 21. The device 1 is also provided with a rechargeable battery 20 and a power supply board 11, to supply a constant voltage to the various components of the device 1. There is also a Wi-Fi radio module 50 for wireless transmission of signals.

The vision module 2 comprises at least one sensor 4 able to acquire an image of a skin lesion L to be analyzed. In one or more embodiments, as well as in the one illustrated in the drawings, such sensor 4 can be made in the form of an RGB camera sensitive in the visible frequencies (430-770 THz), associated to a display screen 23 to visualize the lesion L framed by the camera 4.

The vision module 2 also comprises a lighting system consisting of a plurality of LED devices 5 (also illustrated in FIG. 3A), configured to illuminate the skin area in which the lesion L to be analyzed is present, and at least one polarization filter 6. The term polarization filter means a system aimed at polarizing the incident light which was previously of the non-polarized type, so as to select a single direction of oscillation of the incident electromagnetic wave. This direction depends on the orientation of the polarizer fibers, so electromagnetic waves incident at angles different from this orientation are completely absorbed by the filter.

In one or more embodiments, the device 1 comprises two polarization filters 6 configured with a 90° cross polarization direction. This polarization direction is particularly effective for allowing the visualization of the deeper layers of the skin, eliminating superficial reflections (“glare”) due to the reflection of light on the corneal layer of the patient's skin. In one or more embodiments, the device 1 comprises a single filter associated with a lighting source arranged for the emission of polarized light.

Reference 7 indicates a spacer element associated with the vision module 2, able to define a known distance from the skin area including the lesion L to be analyzed in one or more embodiments of the device 1. Thanks to the arrangement of the spacer element 7 associated with the camera 4, it is possible to acquire an image of the lesion L by placing the device 1 directly in contact with the patient's skin, thus ensuring the maintenance of a fixed distance between the front lens of the focus of the camera 4 and the skin area including the lesion L, in order to ensure a perfect focusing of the framed area. Preferably, this spacer element 7 can be moved axially by means of an adjustment device comprising a threaded coupling and an elastic backlash take up system.

In accordance with a real example of implementation carried out during test experiences performed by the Applicant, camera 4 is equipped with a CMOS sensor with a ⅔″ format and a focus with an overall focal length of 25 mm.

FIGS. 2, 3A, 3B, 4A and 4B illustrate a first embodiment of the invention. With reference in particular to FIGS. 2, 3A and 3B, the vision module 2 is included within a first housing I1. The housing I1 has an “L” shape, with a substantially cylindrical upper portion, protruding from the upper end of a gripping portion 12, also substantially cylindrical, configured and sized to allow an operator to grip the housing I1 and ensure an ergonomic grip during the use of the device 1.

With reference in particular to FIGS. 3A-3B, which respectively represent an assembled perspective view and an exploded perspective view of the first housing I1, in correspondence with the cylindrical portion a slide switch 24 for activating the LED devices 5 and a pair of control buttons 19 are arranged, able to activate the camera 4 and acquire a photo. The vision module 2 including the camera 4 is arranged at an upper portion 13 of the first housing I1. The spacer element 7 and the display screen 23 are arranged at opposite ends of the camera 4. In the exploded view of FIG. 3B, some components of the camera 4 are shown, according to a real embodiment. A printed circuit board 25 of the camera (“camera board”) having dimensions of 30 mm×30 mm houses an RGB CMOS sensor with a ⅔″ format and is connected by means of a FFC-FPC cable 28 to a USB printed circuit board 26 (“USB board”) The latter is configured to produce a video signal in UVC format, which can be transmitted through a USB 3.0 interface.

As indicated above, the device 1 comprises, in addition to the aforementioned vision module 2, also a spectroscopy module 3.

Returning to the schematic view of FIG. 1, it should be noted that the spectroscopy module 3 comprises a spectroscope 8 able to operate in a determined visible-infrared band and to acquire a spectral response of the skin lesion of interest.

In one or more embodiments of the invention, as well as in those illustrated in the drawings, the spectroscopy module 3 further comprises:
a light source 9 able to generate a light signal with a given intensity of reflection, measurable by said spectroscope 8 after the interaction with the skin area including the lesion, and
a connection system 10 for bringing the light emitted by the light source 9 towards the analyzed lesion and the reflected light towards the spectroscope 8.

In the embodiments described herein, the spectroscope 8 is made in the form of a miniaturized spectroscope operating in the visible and near infrared bands. Preferably, the frequency range in which the spectroscope operates is 480 nm-1100 nm.

According to the constructive example shown in FIG. 2, the spectroscopy module 3 is included within a second housing I2 operatively connected to the first housing I1 containing the vision module 2. Such second housing I2 can be made in the form of a prismatic shape instrumentation case comprising at least one handle 100 for transport. According to a real embodiment, such second housing I2 is made with dimensions of 290 mm×260 mm×140 mm.

In other possible embodiments (for example the one illustrated in the following FIG. 6) the two vision 2 and spectroscopy 3 modules can be arranged within the same housing I3, in order to reduce the overall dimensions of the system.

As an alternative to the embodiment of FIG. 2, the portability of the instrument can be ensured by arranging it on a frame, support or device mounted on wheels.

FIGS. 4A, 4B are side views of the housing I2 containing the spectroscopy module 3.

The housing I2 has a USB dual port 14 for connecting respectively a USB connector for camera 4 and the LED module 5, an HDMI port 15 for connecting to an external display screen, an input 16 for connecting to an external power supply network for charging and an ON/OFF switch 18 for turning on/off the spectroscopy module. Furthermore, reference 27 in FIG. 4B indicates a pair of through holes for the passage of the connection system 10 to convey the light emitted by the light source 9 towards the analyzed lesion L and the reflected light towards the spectroscope 8. FIGS. 5A-5B illustrate a plurality of components of the device 1 arranged within the second housing I2.

Preferably, the light source 9 is made in the form of a tungsten halogen lamp (e.g. configured to emit a light signal in the wavelength range between 360 nm and 2400 nm). Alternatively, the light source 9 can be replaced by a set of LED devices such as to cover the spectral sensitivity range of the spectroscope 8 (400-1100 nm).

The connection system 10, to convey the light emitted by the light source 9 towards the lesion L and to convey the light reflected by the lesion L towards the spectroscope 8, can consist of an optical fiber or another optical connection system also composed of suitable optical elements. As shown in the perspective view of FIG. 5A, the optical fiber is a bifurcated optical fiber, comprising two branches 101, 102 respectively connected to the spectroscope 8 and to the light source 9. The two branches 101, 102 are joined to an output branch 103 which constitutes the terminal for the spectroscopy, in such a way that the light signal from the light source 9 is conveyed to the patient's skin framed by the camera 4, and the signal reflected after the interaction with the lesion L of interest is intercepted and conveyed back to the spectroscope 8. It will therefore be appreciated that the signal reaches the head of the fiber (“probe”), that is the output branch 103 which, being connected to the first housing I1, is held in the hand by the doctor during the acquisition of an image of the lesion L at a known distance from the skin and in an orthogonal direction with respect to the latter. The spectroscope 8/light source 9 connection with the respective branch of optical fiber 101, 102 is guaranteed through a SMA-905 interface. As already indicated above, the spectroscopy module further comprises a system which comprises one or more specific optical elements for focusing and dispersion of the light beam to be analyzed.

As previously indicated, the device 1 comprises an electronic control unit E configured for controlling the vision module 2 and the spectroscopy module 3. As shown in the schematic view of FIG. 1, the electronic control unit E is operatively connected to the modules 2, 3, and to the additional sensors 21, and is programmed to allow an operator to manage the aforementioned modules 2, 3 by means of a particularly intuitive graphical interface which can be viewed on the screen 23.

According to the embodiment illustrated in FIG. 5B, preferably, the electronic control unit E is included within the housing I2 together with the spectroscopy module 3. In one or more embodiments, as well as in those illustrated in the drawings, one or more cables (for example USB cables) connect the vision module 2 inside the first housing I1 with the electronic control unit E inside the second housing I2.

In accordance with a real example of implementation carried out during test experiences performed by the Applicant, the electronic control unit E is a control unit equipped with hardware able to run an operating system preferably based on a Linux kernel. It will therefore be appreciated that the unit E represents the core of the device 1 allowing the control of the camera 4 and the spectroscope 8 through a simple user interface on a display, in order to have a particularly intuitive mode of use.

As previously indicated, the device 1 comprises at least a rechargeable battery 20, a power supply board 11 and a control board for the battery recharge process. In one or more embodiments, as well as in those illustrated in the drawings, the rechargeable battery 20 is arranged within the housing I2 (FIG. 5B) and is able to provide a constant voltage of 28 VDC and a power of 75 Ah. The battery 20 is rechargeable through a power supply external to the second housing I2 connected to the electrical network; the connection between the power supply and the battery 20 takes place by means of a 5.5 mm×2.5 mm panel jack connector (indicated with the reference 16 in FIG. 4A), arranged in the rear part of the second housing I2. The power supply board 11 is configured to distribute the power to the various components, by means of various voltage regulators starting from the overall 28 VDC supplied by the battery 20. According to the example illustrated in the drawings, the power supply board 11 is included within the housing I2 and is configured to regulate the voltage as follows:
5 VDC for the power supply of the electronic control unit E;
12 VDC for the power supply of the light source 9 and the screen 23.

Preferably, the device 1 cannot be used if connected to the electrical network, thanks to a decoupling which takes place by means of a suitable hardware isolator integrated in the power supply circuit.

To make the connection between the two housings I1, I2, in the body of the first housing I1 the following wiring (not shown in the drawings) runs, directed to the housing I2:
a first USB cable for the data flow of the camera 4, connected to the USB connector 14, in such a way as to ensure the connection between the electronic control unit E (inside the housing I2) and the camera 4;
a second USB cable still connected to the USB connector 14 for the power supply of the control board 26 of the LED ring 5.

Both cables come out from a single opening on the bottom of the body of the housing I1 and are inserted inside a single protective sheath.

In one or more embodiments, on the other hand, the HDMI connection cable (still not shown in the drawings) between the display 23 and the HDMI port 15 of the housing I2 is external to the body of the housing I1. Alternatively, this connection is internal to the device.

In light of the characteristics described above, in operation, once the data of the lesion L have been acquired by means of the modules 2, 3, the electronic control unit E is configured for storing and sending such acquired data to a processing system SE (illustrated in FIG. 1). The sending of the acquired data from the device 1 to the processing system SE can be carried out, for example, by means of the specific Wi-Fi radio module 50 inserted within the housing I2.

Alternatively, the processing of the data acquired from the lesion L can be performed by means of an internal processing system SI, additional to the control unit E, dedicated to increase its performance for inferential operations.

In one or more embodiments, the processing system SE (or SI) is configured to use automatic data analysis algorithms based on machine learning principles, in order to integrate the data sent by the electronic control unit E and provide the user with an output about the nature of the lesion.

Such processing system SE (or SI) can be based on at least three pre-trained neural networks (to process the RGB image, the spectral response and possibly the data acquired by the other sensors 21), able to provide at least two probabilistic indexes on the malignancy of the lesion. The aforementioned probabilistic indexes can in turn be integrated through a further downstream neural network, producing a single final classification output. In one or more embodiments, the automatic machine learning algorithm operates on an external server.

The final result of the processing can therefore be a single probabilistic index of classification of the lesion L, deriving from the merge of the results of the single automatic classifications related to the spectrum and to the input image, thus obtaining a binary response of high/low risk of the suspicious lesion or a representation of the probability that the lesion belongs to a group of predefined pathological classes.

It will therefore be appreciated that the device 1 is configured to acquire a plurality of input data, in particular one or more images and one or more spectra of the inspected lesion L, based on different physical principles, and to return a single final result of immediate interpretation for the clinical operator or the generic user.

In operation, the electronic control unit E is configured to receive such final result processed by the external processing system SE (or internal SI) and to show said processed result on an external display 22 (FIG. 1). Such external display 22 can be for example made in the form of an external monitor connected to the device 1. Alternatively, the device 1 can comprise an integrated display for viewing the aforementioned processed final result.

Thanks to the above indicated features, the device 1 allows to combine two different physical principles (morphological/visual and biochemical), to acquire different input data on the lesion to be analyzed, making the output classification of the lesion L particularly precise, to support the diagnosis performed by the operator or to provide a first screening instrument on the general public in order to facilitate an early diagnosis of skin lesions.

In one or more embodiments, the processing system of the data acquired by means of the modules/sensors 2, 3 is included within the device 1. In this case, in operation, it is not necessary to set up any external system, since the device 1 internally includes all the components necessary to process the data and provide an informative output result of the state of the lesion. In this case, the electronic control unit E can be configured to process such information and produce a single output result. To enable such processing, the device 1 can be integrated with additional hardware to enhance the computing capacity of the device 1.

In one or more embodiments, as well as in the one illustrated in FIGS. 1, 6, in addition to the vision 2 and spectroscopy 3 modules, the device 1 can comprise further sensor modules 21, based on physical principles different from the vision 2 and spectroscopy 3 modules, in order to further strengthen the precision of the final output classification of the analyzed lesion. Such further sensors 21 can be made in the form of a thermography module 40 including one or more thermal cameras to obtain one or more thermographic images of the lesion and/or an impedance analyzer and/or a vision module operating in the infrared SWIR region (“Short Wave Infrared”) in order to obtain images that emphasize the vascularization of the area including the lesion. The term SWIR refers to the portion of the electromagnetic spectrum in the 1000-2500 nm band.

In one or more embodiments, as well as in the one illustrated in FIG. 6, all the components of the device 1, including the above indicated further sensors 21, can be arranged within a single housing I3. Naturally, the abovementioned electronic control unit E will also be included within the housing I3, so as to allow the management of the different modules/sensors. In the case of such embodiment with a single housing I3, the light source 9 is a miniaturized light source.

As regards the integration of the aforementioned further sensor modules 21, the device 1 comprises an additional hardware group related to the type of sensors 21, in order to allow the correct operation of said further sensor modules 21. For example, in the case of the thermography module, in addition to the thermal camera there will be a hardware heating/cooling system.

Of course, even in the case of a device 1 with two housings I1, I2, operatively connected to each other, such further sensor modules 21 can be added to the vision 2 and spectroscopy 3 modules.

With reference to the embodiment of FIG. 6 including further sensors 21, even in this case, by means of the aforementioned processing system (whether internal or external, anyway not illustrated in the figure), it is provided an integration of the different information acquired by the different modules 2, 3, 21, in particular, one or more images 30 acquired with the vision module 2, a spectral response 31 acquired with the spectroscopy module 3 and, for example, an image 32 acquired with a vision module 21 operating in the SWIR region.

In one or more embodiments in which there is a vision module 21 operating in the SWIR region, the light source 9 is coupled with the optical fiber 10 so as to bring the light signal directly onto the affected lesion. As shown in FIG. 6, by means of one or more optical devices 33 (“beam splitter”) which divide a light beam into several parts, the light reflected by the lesion L is divided into three directions so that it hits the three different sensors 2, 3, 21, respectively sensitive in the bands:
of the visible, in order to produce the RGB image of the lesion L;
of the SWIR, in order to produce the image that emphasizes the vascularization of the lesion;
of the near infrared, in order to generate the reflection spectrum of the lesion.

Also in this case it is provided an integration of the different acquired data by means of an external processing system SE including a downstream neural network able to produce an output on the nature of the lesion.

Also for the embodiment with the aforementioned further sensors 21, the processing system of the data acquired by means of the modules/sensors 2, 3, 21 can be included within the device 1. In this case, in operation, it is not necessary to provide any external system, since the device 1 includes all the components necessary to process data and provide an informative output result on the state of the lesion. In this case, the electronic control unit E can be configured to process such information and produce a single output result. To allow such processing, the device 1 can be integrated with additional hardware to enhance the computing capacity of the device 1. This embodiment in which the processing system is included within the device 1 is also applicable to the case in which the device 1 only includes the vision 2 and spectroscopy 3 modules, without including the aforementioned further sensors 21.

FIGS. 7, 8A and 8B are variations of FIGS. 2, 3A and 3B which illustrate yet another embodiment. In these figures the parts which are common to those illustrated in FIGS. 2, 3A and 3B are indicated with the same reference numbers.

With reference to FIG. 7, in the case of this example the screen 23 is associated with the housing I2, instead of the housing I1. Furthermore, in order to ensure redundancy and a further alternative for using the device on the basis of user comfort, a pair of buttons 19, having the same functions above described, is also housed on the support 45 of the display 23, which in this example is obtained with 3D printing technology.

FIG. 7 shows the two optical fibers 101, 102 protruding from the housing I2 and converging into a single optical fiber 103 whose distal end is provided with a gripping portion 104.

With reference to FIG. 8A, in this example the housing I1 has a general external conformation not very different from that of FIGS. 2 and 3A, except for the elimination of the display 23.

As can be seen in FIG. 8B, the components inside the housing I1 include a lighting sub-module m1 which comprises a printed circuit board 20′ of the lighting system of the vision module, connected to the control board 26 of the module itself by means of a FFC-FPC cable 27′, the polarization filters 6 and 3D printed supports 22′.

An optical sub-module m2, again illustrated in FIG. 8B, includes a lens system 24′ and a printed circuit board 25 of the camera (“camera board”) having dimensions of 30 mm×30 mm, which houses an RGB CMOS sensor with a ⅔″ format and is connected by means of an FFC-FPC cable 28 to an USB printed circuit board 29 (“USB board”). The latter is configured to produce a video signal in UVC format, which can be transmitted through a USB 3.0 interface.

Therefore an electronic control sub-module m3 is configured, comprising the control board 26 of the vision module and the USB printed circuit board 29 functional for the control of the camera.

In this example, a first USB cable 34 connects the electronic unit E to the USB printed circuit board 29 (“USB board”) and a second USB cable 35 connects the electronic control unit E to the control board 26 housed in the vision module, suitably separated from the printed circuit board 29 by four spacer elements. Such control board 26 manages the control of the illuminator, if it is implemented in the form based on LED elements.

FIGS. 9-13 refer to an embodiment of the device 1 according to the invention, in which the further sensors 21 take the form of a thermography module 40.

As will be evident from the following description, the operation of the thermography module 40 is based on the difference in terms of thermal properties (thermal conductivity and thermal diffusivity) which arise between a healthy tissue and a tumor tissue. More precisely, with the growth of the tumor tissue, angiogenesis and an increase in local blood flow occur, which determine a change in the thermal properties of the tissue and an increase in the local production of heat.

In accordance with the schematic view of FIG. 9, the thermography module 40 comprises a thermal sub-module 41, a thermal interaction instrument 42 for carrying out a thermal stimulation of the tissue to be investigated and a power supply sub-module 43.

With reference to FIGS. 10A, 10B, the power supply sub-module 43 is arranged to supply the power necessary for the operation of the thermal sub-module 41 and the thermal interaction instrument 42. The power supply sub-module 43 can be made in the form of a case 44 (for example of metallic material) which can comprise one or more handles to facilitate transport.

FIG. 10B illustrates the components integrated within the case 44, comprising:
at least one battery 45, preferably lithium-ion;
an ethernet switch 46 able to create a local LAN network;
a power distribution board 47 for supplying the components of the thermography module 40;
an external panel connector 48 designed for the connection of the thermal interaction instrument 42;
a DC-DC converter 49 for supplying the thermal sub-module 41;
at least one ethernet connector 50′ designed for interfacing with an external electronic device;
at least one fan 51 designed for cooling the components;
an on/off button 52.

FIGS. 11A, 11B illustrate a preferred embodiment of the thermal interaction instrument 42 for carrying out a thermal stimulation of the tissue to be investigated. The instrument 42 is able to cool down a skin area containing the lesion L to be analyzed and the surrounding skin for a determined period of time (for example 60 seconds).

With reference to FIG. 11A, the thermal interaction instrument 42 has a housing 53 comprising an operating head 54 and a handle 55 to facilitate the use of the instrument 42. An on/off button 71 and a display screen 72 to display information and/or to adjust some functionalities of the instrument 42 are provided close to the operating head 54.

With reference to the embodiment illustrated in FIG. 11B, the thermal interaction instrument 42 internally comprises the following components:
a power supply connector 56 designed for connection to the power supply sub-module 43;
a Peltier device 59 able to cool down the contact region between the operating head 54 and the skin region to be analyzed. It should be noted that the operation of the device 59 is based on the Peltier effect whereby the application of a voltage on two faces of the device 59 generates a thermal gradient between the faces themselves;
a printed circuit board 57 for controlling the instrument 42 comprising a micro-controller 58 able to read the contact temperature with the skin by means of a temperature sensor (for example a group of thermistors 58′) and to regulate the current supplied to the device 59 implementing a negative feedback control. In one or more embodiments, the contact temperature to be reached is 15° C.;
an interface contact plate 60 with the subject's skin. Preferably, the plate is made of aluminum and is connected in a removable way to the housing 53 to allow the disinfection of the plate between different measurements,
a heat sink 61 able to keep the face of the device 59 at a constant temperature;
at least one fan-type cooling device 62 to dissipate the generated heat towards the outside, so as to avoid excessive heating of the components inside the instrument 42.

FIGS. 12A, 12B illustrate a preferred embodiment of the thermal sub-module 41. The thermal sub-module 41 comprises at least one thermal camera 63 able to record the absolute temperature of the area framed by the thermal camera 63, for example with a sensitivity of 65 mK. Preferably, the acquisition frequency is 1 Hz. As illustrated in FIG. 12A, the sub-module has an external structure 64 designed to be gripped by an operator, to allow a user-friendly usage. The thermal sub-module 41 comprises the following components:
an on/off button 66;
an RGB camera 67 sensitive to visible light, able to locate the position of the lesion within the thermal image detected by the thermal camera 63;
an internal controller 65 able to control the thermal camera 63 and the RGB camera 67.

In operation, at the data acquisition start time t=0, the cooled area appears completely uniform in the thermal image detected by the thermal camera 63, making it almost impossible to identify the lesion within it. Consequently, it is possible to use two different methods to trace back to the position of the lesion L to be analyzed:
use at least one identification marker element, preferably an aluminum adhesive tape positioned around the lesion L. It should be noted that aluminum has a very low emissivity and is always visible throughout the acquisition, thus allowing to locate the position of the lesion in each detected image;
overlap the thermal image and an image acquired with the RGB camera 67, in order to identify the position of the lesion by means of this image overlapping, since the RGB image is likely to acquire the position of the lesion L.

FIG. 13 illustrates compared temperature profiles 68, 69, respectively of an acquisition performed on a healthy tissue and of an acquisition performed on a tissue with a suspicious lesion L, by using the device 1 comprising the thermography module 40. Thanks to the aforementioned comparison of the general trends of profiles 68, 69, it is possible to quickly and reliably trace back to objective information on the nature of the analyzed lesion L.

In one or more embodiments of the invention, the thermography module 40 is integrated in the device 1, together with the vision and spectroscopy modules 2, 3 (see in FIG. 1 the block “other sensors” indicated with the number 21). In this case, the processing system SE, SI (external or internal to device 1) is able to process the data acquired by all the aforementioned modules, so as to provide a single output result particularly significant of the skin lesion L of interest. The output result can be probabilistic in relation to the malignancy of the lesion.

In one or more embodiments of the invention, with reference to the input data acquisition modules, the device 1 to support dermatological diagnosis support for the recognition of a skin lesion L comprises only a thermography module 40 according to one or more of the features previously described, without the use of additional modules (for example vision and spectroscopy 2, 3). In this case, the thermography module 40 can be connected to an external processing system SE (for example an electronic computer) for processing the acquired data. Still in the case in which only the thermography module 40 is present (without vision 2 and spectroscopy 3 module), it can be integrated within the device 1 together with a control unit E and an internal processing system SI, according to the architecture illustrated in FIG. 1.

In one or more embodiments, the control unit E of the device 1 is configured for:
controlling the acquisition of temperature profiles describing the thermal recovery of skin regions which are cooled by means of the thermal interaction instrument 42;
sending the acquired data to the processing system SE, SI,
automatically comparing the thermal recovery profiles of a healthy skin region and of a region containing the lesion of interest;
automatically processing an output result representative of objective information about the characteristics of the skin lesion of interest.

In one or more embodiments, the thermal acquisition is managed through an HMI man-machine interface visible by the operator. The HMI interface can be arranged on the housing I1, I2 of device 1 (screen 23 of FIGS. 2, 3A, 3B, 7) or on an external display 22 (FIG. 1) connected to the processing system SE.

In light of the previous description, the analysis process according to the use of a device 1 comprising a thermography module 40, comprises the following steps:
thermally stimulating the area affected by the lesion L by means of the thermal interaction instrument 42. Preferably, the stimulation step involves a local cooling for 60 seconds;
recording the thermal recovery of the previously cooled region, by means of the thermal camera 63, preferably for a time of 180 seconds;
automatically comparing the general trend of thermal recoveries of a cooled healthy skin region and of the region affected by the lesion to be analyzed;
if the thermal recoveries are statistically different, generating an alert to a clinical operator who is employing the technology. It should be noted that in order to maximize the reliability of the process, the acquisition step of the thermal recovery data can provide successive consecutive measurements alternated by a rest time, both of the healthy tissue and of the region with the lesion L.

The scalability of all components used in the above described embodiments, allows the realization of one or more embodiments of the device such as to be ancillary and complementary to the use of mobile devices (“smartphones”) used in everyday life by any person and which can already integrate one or more of the above described components, in particular the vision module.

Thanks to the above indicated features, the device 1 according to the invention achieves a series of important advantages, including:
guaranteeing objective and quantitative information on the analyzed skin lesion, in order to obtain an extremely precise and reliable characterization of the analyzed lesion;
providing a single particularly significant output on the malignancy of the analyzed lesion starting from different inputs based on different physical principles; and
allowing a user-friendly usage for the doctor who uses the device to analyze the lesion of interest, or for the general user who uses the device as a first monitoring instrument of his/her condition with a view to spreading telemedicine and implementing a participatory, personalized and preventive medicine, particularly through the integration with cloud storage mechanisms of the clinical data of each user.

Naturally, the principle of the invention remaining the same, the constructive details and the embodiments may vary widely with respect to those described and illustrated purely by way of example, without thereby departing from the scope of the present invention.

1. A device (1) to support dermatological diagnosis for a recognition of a skin lesion (L), including to evaluate a nature of a skin lesion (L), said device comprising:
a vision module (2) including at least one sensor (4) configured to acquire an image of the skin lesion (L) of a patient,
a spectroscopy module (3) including at least one spectroscope (8) configured to acquire a spectral response of the skin lesion (L) in a determined visible and infrared band,
at least one housing (I1, I2, I3) including said vision module (2) and said spectroscopy module (3),
at least one electronic control unit (E) configured for:
controlling said vision module (2) and said spectroscopy module (3),
storing a plurality of data acquired by means of said vision and spectroscopy modules (2, 3), including one or more images acquired by means of said vision module (2) and one or more spectral responses acquired by means of said spectroscopy module (3),
sending said plurality of acquired data to a processing system (SE, SI),
receiving an objective evaluation result of the skin lesion (L) processed by said processing system (SE, SI), as a result of an integration of the plurality of acquired data performed by said processing system (SE, SI), and
showing said objective evaluation result on a display screen (22, 23) in such a way that said device (1) is configured to acquire by means of said vision module (2) and said spectroscopy module (3) a plurality of input data about the skin lesion (L) to be analyzed, based on different physical principles, and to provide a visualization of a single output result representative of objective information about characteristics of the skin lesion (L) to be analyzed,
wherein said at least one housing comprises:
a first housing (I1) including said vision module (2) therewithin and provided with a gripping portion (12) which is configured to ensure an ergonomic grip by an operator during use of the device (1),
a second housing (I2) connected to the first housing (I1) and including therein said spectroscopy module (3) and said at least one electronic control unit (E),
wherein the spectroscopy module (3) comprises an optical connection system (10), to convey light emitted by a light source (9) towards the skin lesion (L) and to convey light reflected by the skin lesion (L) towards the at least one spectroscope (8), the optical connection system (10) comprising two optical fiber branches (101, 102) respectively connected to the at least one spectroscope (8) and to the light source (9), the two optical fiber branches (101, 102) converging into a single optical fiber (103) whose distal end is provided with a gripping portion (104).

2. The device (1) according to claim 1, wherein said at least one sensor (4) is an RGB camera sensitive in the visible frequencies (430-770 THz), and configured to acquire the image of the skin lesion (L).

3. The device (1) according to claim 1, wherein said vision module (2) further comprises:
a lighting system consisting of a plurality of LED devices (5) configured to illuminate an area of the patient's skin including the skin lesion (L) to be analyzed, and
at least one polarization filter (6) configured to provide for a visualization of deepest layers of the patient's skin, eliminating surface reflections due to reflection of light on a corneal layer of the patient's skin.

4. The device (1) according to claim 3, comprising two polarization filters (6) configured with a cross polarization direction at 90° between them.

5. The device (1) according to claim 1, wherein a spacer element (7) is associated with said vision module (2), said spacer element (7) configured to define, during use of the device (1), a known distance from an area of the patient's skin including the skin lesion (L) to be analyzed, in order to ensure a focusing of the area framed by said at least one sensor (4).

6. The device (1) according to claim 5, wherein said spacer element (7) is configured to be moved axially by means of an adjustment device comprising a threaded coupling and an elastic backlash take up system.

7. The device (1) according to claim 3, wherein said spectroscopy module (3) further comprises:
a system comprising one or more specific optical elements for focusing and dispersion of the light signal to be analyzed.

8. The device (1) according to claim 7, wherein said light source (9) of the spectroscopy module (3) is integrated with said lighting system of the vision module (2).

9. The device (1) according to claim 1, wherein said at least one electronic control unit (E) is configured to control said vision and spectroscopy modules (2, 3) by means of a graphic interface viewable on the display screen (23) included in said at least one housing (I1, I2, I3).

10. The device (1) according to claim 1, wherein said processing system (SE) is arranged externally to said at least one housing (I1, I2, I3).

11. The device (1) according to claim 1, wherein said at least one electronic control unit (E) is further configured to process said data acquired by means of said vision and spectroscopy modules (2, 3) with help from said processing system (SI), arranged internally to said at least one housing (I1, I2, I3), and to produce a processed result containing objective information about the characteristics of the skin lesion (L) to be analyzed.

12. The device according to claim 11, wherein said electronic control unit (E) is further configured to process said acquired data on a basis of machine learning algorithms, in order to integrate data sent by the electronic control unit (E) and to produce an objective classification of a nature of the analyzed skin lesion (L).

13. The device (1) according to claim 1, comprising further sensors (21), based on physical principles different from those of the vision and spectroscopy modules (2, 3), so as to strengthen a precision of a final output classification of the skin lesion (L),
wherein said further sensors (21) comprise a thermography module (40),
wherein said at least one electronic control unit (E) is configured for:
controlling said further sensors (21), said vision module (2) and said spectroscopy module (3),
storing a plurality of data acquired by means of said vision and spectroscopy modules and said further sensor (2, 3, 21),
sending said plurality of acquired data to the processing system (SE, SI),
receiving an objective evaluation result of the skin lesion (L) processed by said processing system (SE, SI), as a result of the integration of the plurality of acquired data performed by said processing system (SE, SI) and
showing said objective evaluation result on the display screen (22, 23).

14. The device (1) according to claim 13, wherein said further sensors (21) further comprise one or more of the following modules: an impedance analyzer, another vision module operating in a short wave infrared region in order to obtain images that emphasize vascularization of an area including the skin lesion.

15. (canceled)

16. (canceled)

17. The device (1) according to claim 1, wherein said device (1) defines a single housing (I3) incorporating both the vision module (2) and the spectroscopy module (3).

18. The device (1) according to claim 13, wherein said thermography module (40) includes:
at least one thermal interaction instrument (42), configured to cool down a skin region comprising the skin lesion (L) to be analyzed; and
at least one thermal camera (63), configured to obtain one or more thermographic images of the skin lesion (L) to be analyzed.

19. The device (1) according to claim 18, wherein said thermal interaction instrument (42) has a housing (53) comprising an operating head (54) and a handle (55) and a Peltier device (59) configured to cool down a contact region between the operating head (54) and the skin region to be analyzed.

20. The device (1) according to claim 19, wherein said operating head (54) comprises an interface contact plate (60) connected in a removable way to the housing (53) to provide for disinfection between different measurements.

21. The device (1) according to claim 18, wherein said at least one electronic control unit (E) is configured for:
controlling an acquisition of temperature profiles describing a thermal recovery of a healthy skin region and of the skin region containing the skin lesion (L), both cooled down by means of the thermal interaction instrument (42);
sending the acquired data to the processing system (SE, SI),
automatically comparing said temperature profiles; and
automatically processing an output result representative of the objective information about the characteristics of the skin lesion of interest.

22. A system to support dermatological diagnosis for the recognition of skin lesions (L), comprising the device (1) according to claim 1 including the processing system (SE, SI) configured to receive the data acquired by said device (1) and to process a final evaluation result of the skin lesion analyzed.
